(19) United States
(12) Patent Application Publication
(10) Pub. No.: US 2006/0062849 A1
(43) Pub. Date: Mar. 23, 2006

(54) Oral formulation of creatine derivatives and method of manufacturing same

(75) Inventor: Edward A. Byrd, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: Medical Research Institute

(21) Appl. No.: 10/947,058

(22) Filed: Sep. 21, 2004

(51) Int. Cl. A61K 9/22; A61K 31/22

(52) U.S. Cl. 424/468; 514/551

(57) ABSTRACT

Oral formulation of creatine derivative and in particular creatine esters and more particularly ethyl esters of creatine are described. The formulations comprise a phosphate such as dicalcium phosphate, a biodegradable polymer such as a polyvinyl pyrrolidine and a starch. The formulation may further comprise other excipients such as metal salt of a stearate, e.g. magnesium stearates. The formulation is produced as flowable particles with a sieve size of about 20 to 60 which particles are coated with a shellac to mask taste, avoid moisture uptake, and extend shelf life.

### FIELD OF THE INVENTION

The invention relates generally to the field of nutritional supplements and more particularly to oral formulations of creatine derivatives.

### BACKGROUND OF THE INVENTION

Creatine is an endogenous nutrient produced naturally by the liver in most vertebrates. The uses of creatine are many, including use as a supplement to increase muscle mass and enhance muscle performance as well as in emerging applications in the treatment of neuromuscular disorders.

Creatine, or N-(aminoiminomethyl)-N-methylglycine, is a sarcosine derivative present in the muscle tissue of many vertebrates, including man. Creatine is a central component of the metabolic system, and is involved in the provision of energy for work and exercise performance. Phosphocreatine (also known as creatine phosphate and phosphoryl creatine) helps to regenerate Adenosine TriPhosphate (ATP) during short bursts of high intensity exercise, and it has been found that the depletion of phosphocreatine has been associated with the onset of fatigue. It has also been discovered that the phosphocreatine pool in skeletal muscle is expandable. This has led to the oral supplementation of creatine and phosphocreatine to increase the levels of these components in muscle, to thereby enhance exercise performance during intermittent activities that require strength and power. WO 94/02127, published on Feb. 3, 1994, discloses the use of creatine, optionally combined with amino acids or other components, in order to increase the muscle performance in mammals.

Creatine is synthesized from amino acids in the liver, pancreas and kidney, by the transfer of the guanidine moiety of arginine to glycine, which is then methylated to form creatine. Creatine which is synthesized in the liver, pancreas and kidney, is released into the bloodstream and actively taken up by the muscle cells, using the Na+ gradient. Creatine oral supplementation has been used to increase creatine and creatine phosphate stores, which are needed for high energy phosphorus metabolism. Recovery after high intensity exercise involves a resynthesis of phosphocreatine, which occurs via an oxygen-dependent process with half-life of about 30 seconds. During short-term high intensity intermittent exercise, the active muscles rely heavily on phosphocreatine for production of ATP. The rate of phosphocreatine resynthesis can be accelerated by the use of creatine supplementation in subjects who demonstrated an increase in creatine concentration. The benefits of creatine supplementation are particularly evident in high intensity activities that are intermittent in nature.

The creatine transport protein has an increased affinity for creatine and concentrates creatine within the cell. Once inside the cell, very little creatine is lost (approximately 2 grams per day in a 70 kg male). Based upon this information, it follows that small increases of plasma creatine (which can occur with creatine supplementation) result in increased transport activity. The loss of creatine from skeletal muscle is typically about 3% per day, which closely matches the amount of creatinine non-enzymatically produced by living human muscle. The main mechanism by which creatine is lost, is the conversion of creatine to creatinine, which is an irreversible non-enzymatic process. Thus, creatine lost from a cell is considered to be negligible, and the concentration of creatine in the cell is not at risk of depletion by virtue of exercise. Thus, the main advantage of creatine administration is in the fact that cellular creatine concentration is stable and not prone to being lost.

The most commonly used creatine supplement for oral consumption, is creatine monohydrate. Body builders find that shortly after beginning the use of creatine as a nutritional supplement, muscles take on additional mass and definition. Thus creatine supplements are becoming more popular as a steroid-free means of improving athletic performance and strength. Increasing the creatine in a diet through supplementation may therefore be useful to increase the blood plasma level of creatine and thus increase the amount of creatine in the muscles.

Creatine monohydrate is most commonly sold as a nutritional supplement in powder form. The powder may be blended with juices or other fluids, and then ingested. Prompt ingestion is important, because creatine is not stable in acidic solutions, such as juices. If creatine is retained in acidic solutions for even relatively short periods of time, most or all of the creatine in this solution converts to creatinine, which does not have the beneficial effects of creatine.

Creatine monohydrate supplementation at a dosage of 20 grams per day for a 5 day period has been the standard used during most studies in humans. Conventionally, creatine monohydrate is dissolved in approximately 300 milliliters of warm to hot water, the increased water temperature thereby increasing the solubility of creatine monohydrate. It has been found that creatine is not decomposed in the alimentary tract after oral administration, since there is no appreciable increase in urinary urea or ammonia. The results obtained for the conversion of retained creatine to creatinine have led researchers to believe that creatine is completely absorbed from the alimentary tract, then carried to the tissues, and hence either stored in the tissues or immediately rejected and eliminated by way of the kidneys.

Another problem with existing creatine supplementation is in the ability to provide consistent uniform results. It is believed that these inconsistent results arise because of the current methods of delivering creatine to the human body area. Current creatine oral supplementation, as discussed above relies on the use of creatine in powder form, which is dissolved in water and then taken orally. However, creatine in powder form does not dissolve well in water or other neutral pH liquids. The solubility of creatine in water is low, about 1 g in 75 ml. To obtain 10 grams, a subject would have to consume almost a liter of liquid. While increasing the temperature of the water increases the solubility of creatine monohydrate, there still is no consistency in the amount of creatine that is effectively dissolved in the water. For this reason, the consumer will take in varying amounts of creatine when consuming creatine monohydrate powder dissolved in water or other liquids.

Typically, creatine is taken up into muscle cells by specific transport proteins, the creatine transporter, and converted to phosphocreatine by creatine kinase. Muscle cells, including skeletal muscle and the heart muscle, function by utilizing cellular energy released from the conversion of adenosine triphosphate (ATP) to adenosine diphosphate (ADP). The amount of phosphocreatine in the muscle cell determines the amount of time it will take for the muscle to recover from activity and regenerate adenosine triphosphate (ATP). Phosphocreatine is a rapidly accessible source of phosphate required for regeneration of adenosine triphosphate (ATP) and sustained use of the muscle.

For example, energy used to expand and contract muscles is supplied from adenosine triphosphate (ATP). Adenosine triphosphate (ATP) is metabolized in the muscle by cleaving a phosphate radical to release energy needed to contract the muscle. Adenosine diphosphate (ADP) is formed as a byproduct of this metabolism. The most common sources of adenosine triphosphate (ATP) are from glycogen and creatine phosphate. Creatine phosphate is favored as a ready source of phosphate because it is able to resynthesize adenosine triphosphate (ATP) at a greater rate than is typically achieved utilizing glycogen. Therefore, increasing the amount of creatine in the muscle increases the muscle stores of phosphocreatine and has been proven to increase muscle performance and increase muscle mass.

However, creatine itself is poorly soluble in an aqueous solution. Further, creatine is not well absorbed from the gastrointestinal (GI) tract, which has been estimated to have a 1 to 14 percent absorption rate. Thus, current products require large amounts of creatine to be administered to be effective, typically 5 grams or more. Additionally, side effects such as bloating, gastrointestinal (GI) distress, diarrhea, and the like are encountered with these high dosages.

Therefore, it would be desirable to provide an improved approach for enhancing absorption of creatine.

### SUMMARY OF THE INVENTION

Oral formulations of a creatine derivative and in particular creatine esters and more particularly ethyl esters of creatine are described. The formulations comprise a phosphate such as dicalcium phosphate, a biodegradable polymer such as a polyvinyl pyrolidine and a starch. The formulation may further comprise other excipiants such as a metal salt of a stearate, e.g. magnesium stearates. The formulation may be a controlled release formulation. Methods of the invention include methods of making oral dosage forms of the formulation and methods of treatment using those oral dosage forms.

An aspect of the invention is that the composition of the formulation is flowable making it possible to create tablets, caplets, capsules and the like in an efficient manufacturing process.

Another aspect of the invention is to provide a creatine derivative formulation with a particle size which allows for freely flowable particles.

Still another aspect of the invention is to provide for a formulation of flowable particles which are readily compressable into tablets in a tablet manufacturing process.

Yet another aspect of the invention is to provide an oral formulation of a creatine derivative with enhanced bioavailability of active compound relative to an equivalent creatine formulation.

Another aspect of the invention is that the creatine derivative in the formulation is a coated in a manner so as to mask taste and to minimize exposure to water.

Yet another aspect of the invention is to increase the bioavailability of the creatine to a patient subject.

Still another aspect of the invention is to provide a controlled release formulation of a creatine derivative.

Yet another aspect of the invention is to provide a method of enhancing the muscle performance of a subject by regularly administering to the subject a therapeutically effective amount of a creatine derivative in a formulation of the invention.

Still yet another aspect of the invention is to provide a method of treating a neuromuscular disorder of a subject by regularly administering to the subject a therapeutically effective amount of a creatine derivative in a formulation of the invention.

Another aspect of the invention is to provide a method of increasing the percentage of muscle tissue and decreasing the percentage of fat tissue of a subject by regularly administering to the subject a therapeutically effective amount of a creatine derivative in a formulation of the invention.

Yet another aspect of the invention is to provide a method of increasing muscular endurance of a subject by regularly administering to the subject a therapeutically effective amount of a creatine derivative in a formulation of the invention.

Another aspect of the invention is to provide an oral formulation comprising a creatine derivative, a dicalcium phosphate, a biodegradable polymer, and a starch in proportions such that the formulation is flowable and formable into an oral dosage unit.

Still another aspect of the invention is to provide such an oral formulation comprising a creatine ethyl ester, a dicalcium phosphate, a biodegradable polymer, such as a polyvinyl pyrolidine, a starch and a metal salt of a stearate such as a magnesium stearate.

Yet another aspect of the invention is to treat patients by the administration (e.g. BID, TID) of an oral dosage unit of the invention so as to maintain therapeutic levels of creatine in the patient over long periods each day (e.g. 4 hours of more,) for 5 days or more, 10 days or more or 30 days or more.

Another aspect of the invention is to provide oral dosage units with improved shelf life.

Still another aspect of the invention is to provide a formulation which substantially eliminate water absorption prior to ingestion by the patient.

These and other objects, aspects, advantages, and features of the invention will become apparent to those persons skilled in the art upon reading the details of the invention as more fully described below.

### BRIEF DESCRIPTION OF THE DRAWINGS

FIG. 1 is a graph of percent of particles versus sieve size for two different formulations of the invention.

### DETAILED DESCRIPTION OF THE INVENTION

Before the present, formulations, methods and components used therein are disclosed and described, it is to be understood that this invention is not limited to particular compounds, excipients or formulations as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided are subject to change if it is found that the actual date of publication is different from that provided here.

### Definitions

The term “creatine” refers to a compound having the following structural formula:

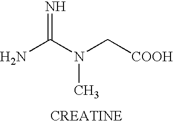

Further, unless specified otherwise the term covers pharmaceutically acceptable salts (e.g. Na and K salts) of the acid wherein the COOH is COONa. Thus, in the above structure the sodium salt is when COOH becomes COONa. In referring to pharmaceutically acceptable salts the term is intended to encompass a conventional term of pharmaceutically acceptable acid addition salts which refer to salts which retain the biological effectiveness and properties of the free-base form of the acid and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malconic acid, succinic acid, maleic acid, fumaric, tartaric acid, citric acid, benzoic acid, cinnamic acid, mandelic acid, methanesulfonic acid, ethanesulfonic acid, p-toluenesulfonic acid, salicylic acid and like forms which can be formed and maintain biological effectiveness and not have significant undesirable biological properties.

The term “creatinine” refers to a compound having the following structure:

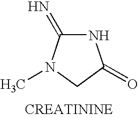

The term “excipient material” is intended to mean any compound forming a part of the formulation which is intended to act merely as a carrier, i.e., not intended to have biological activity itself beyond that of regulating release of a biologically active component.

The term “creatine derivative” refers to a compound having the following structure: 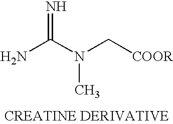
wherein R is not hydrogen but is hydrocarbyl.

The term “hydrocarbyl” is used herein to include substantially hydrocarbyl groups as well as purely hydrocarbyl groups. The description of these groups as being substantially hydrocarbyl means that they contain no non-hydrocarbyl substituents or noncarbon atoms which significantly affect the hydrocarbyl characteristics or properties of such groups relevant to their uses as described herein. Non-limiting examples of substituents which do not significantly alter the hydrocarbyl characteristics or properties of the general nature of the hydrocarbyl groups of this invention include the following:
Alkyl including those comprising one to twenty carbons including lower alkyl e.g. methyl, ethyl, butyl, isobutyl, tertiary butyl, etc.
Alkenyl including those comprising one to twenty carbons and lower alkenyl.

The term “lower” as used in the present specification and claims, when used in conjunction with terms such as alkyl, alkenyl, alkoxy, and the like, is intended to describe such groups which contain a total of up to 7 carbon atoms.

The term “chemical degradation” is intended to mean that the creatine active ingredient is subjected to a chemical reaction which disrupts its biological activity.

The term “particle size” refers to the size of particles of formulation of a creatine derivative of the invention. The particle size is based on United States mesh size ranges. Mesh sizes are defined by the mesh size of sieves used to separate particles. Sieve sizes may be graduated and defined by the number of lines per inch of each sieve e.g. 50 lines per inch or 20 lines per inch. Size specifications are designated by organizations such as ANSI and FEPA. Indicating a size of 30/40 U.S. mesh means that most of the particles in the formulation would fall between 30 mesh and the 40 mesh sieve. Standards permit a small amount of oversize and undersize materials. However, the undersized materials generally range to 2 to 4% as do the oversize materials. In formulating a creatine derivative formulation of the invention it has been found that a formulation which is processed so that the particles would fall between a sieve 18 and sieve 60, or a sieve 20 and a sieve 40 can be made flowable and the flowable material can be compressable into a tablet in accordance with the invention. A sieve 18 has a sieve opening of 1,000 microns, sieve 20 has an opening of 841 microns; sieve 25 has an opening of 707 microns; sieve 30 has an opening of 595 microns; sieve 35 has an opening of 500 microns; sieve 40 has an opening of 420 microns; sieve 45 has an opening of 354 microns; sieve 50 has an opening of 297 microns; sieve 60 has an opening of 250 microns.

The terms “treating” and “treatment” and the like are used herein to generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease and/or its symptoms or conditions. The invention is directed towards treating a patient so as to result in any enhancement of muscle performance, building muscle tissue, treating a neuromuscular disorder, improving muscle endurance or reducing fat tissue. Formulations of the invention may be administered to patients having myoclonus (i.e., a neuromuscular disorder characterized by the occurrence of irregular, asynergic, and jactitious contractions of muscles producing non repetitive, brief, involuntary movements in various body areas) as a symptom of epilepsy, neurodegenerative disease such as Parkinson's disease, multiple sclerosis or amyotrophic lateral sclerosis (ALS) and Tourette's syndrome.

The term “quick release formulation” refers to a conventional oral dosage formulation. Such a formulation may be a tablet, capsule or the like designed to provide for substantially immediate release of the active ingredient e.g. a creatine ethyl ester and includes enteric coated oral formulations which provide some initial protection to the active ingredient and thereafter allow substantially immediate release of substantially all the active ingredient. A quick release formulation is not formulated in a manner so as to obtain a gradual, slow, or controlled release of the active ingredient.

### Formulation in General

The formulation of the invention is preferably an oral dosage formulation which may be in any suitable oral form including tablets, capsules, caplets, suspensions, etc. The dosage may be of any desired size in terms of the creatine ester active ingredient. However, sizes in a range of about 200 mg to about 5,000 mg are generally used, and are preferably in the range of about 500 mg to about 1,000 mg and more preferably about 750 mg±10%. The amount a patient will need to obtain an optimum therapeutical effect will vary with a number of factors known to those skilled in the art, e.g., the size, age, weight, sex and condition of the patient. The patient may begin with daily doses of about 500 mg and determine, for example, if muscle endurance is enhanced. If the desired results are not obtained in one week, the daily dosage amount can be increased in increments of 100 to 300 mg/day up to any useful amount, e.g., 5,000 mg/day. A suggested dosage is to administer two 500 mg tablets in the morning and administer one 500 mg tablet four hours later and repeat daily over five or more days. The larger initial dosage has been found effective in obtaining a desired effect which after being obtained can be maintained by a lower dose. Thus, a biological system may be “kick started” by a high therapeutic level and then maintained at a lower level which is also therapeutic in terms of obtaining a desired result.

A typical formulation contains about 70-90% by weight creatine active ingredient with the remainder being excipient material. Preferably the formulation comprises 75% to 85% active ingredient or about 80%±10% active ingredient by weight. Thus, a particularly preferred oral formulation of the invention comprises about 800-1000 mg±10% of creatine and about 200 mg±10% of excipient material. Human patients generally eat during the day and sleep at night. Eating causes increased glucose levels. Accordingly, it is generally preferable to give a larger dose of creatine at the beginning of the day. This may include two 500 mg tablets or a single 1,000 mg tablet. Later in the day (about 4 hours later) the patient will take an additional 500 mg for a typical daily dose of about 1,500 mg for a 70 kg man.

One embodiment of the formulation is characterized by (a) protecting the active ingredient from chemical degradation in a patient's gastrointestinal tract and (b) releasing the active ingredient in a controlled manner. By gradually releasing the active ingredient, the serum levels of creatine obtained are (1) lower than those obtained with a single non-controlled release formulation; and (2) maintained over longer periods of time at a therapeutic level than obtained with a single non-controlled release formulation. Specifically, a formulation of the invention releases active ingredient so as to obtain a blood serum level in a human patient in a range of about 50 to about 300 micrograms/ml of plasma. The range is preferably about 75 to about 125 micrograms/ml of plasma and more preferably about 115 micorgrams/ml of plasma ±5%.

Creatine ethyl ester is characterized as (1) non-toxic at relatively high levels, i.e., levels well in excess of therapeutic levels; and (2) metabolized by human patients to the same metabolites as creatine. The present invention relies in part on the discovery that creatine esters provide desirable therapeutic results even at very low levels provided those low levels are maintained over an extended period of time; whereas therapeutic results are not obtained (even with higher levels) if the therapeutic level is not maintained over a sufficient period of time. Further, the present invention relies in part on the discovery that therapeutic results are further improved if the formulation is delivered over a period of five or more days, preferably thirty or more consecutive days with long periods of therapeutic levels of creatine being obtained on each of the days.

### Formulating Particle Size

Creatine ethyl ester is formulated in accordance with the formula put forth below:

| | |
|---|---|
| creatine ethyl ester | 83% |
| Di-Calcium Phosphate | 10% |
| polyvinyl pyrrolidone (Kollidon 90) | 3% |
| Starch | 3% |
| Magnesium stearate | 1% |
| TOTAL | 100% |

Two different batches of the formulation were created and produced into two different particles sizes which are shown below as “A” and “B” in the following Table 1.

| A | B | |
|---|---|---|
| Percent | Percent | Sieve |
| 51.8 | 9.3 | 20 |
| 2.8 | 27.6 | 40 |
| 10.4 | 17.0 | 60 |
| 4.7 | 9.3 | 80 |
| 5.6 | 105 | 140 |
| 4.8 | 7.2 | 200 |
| 0.6 | 19.1 | PAN |

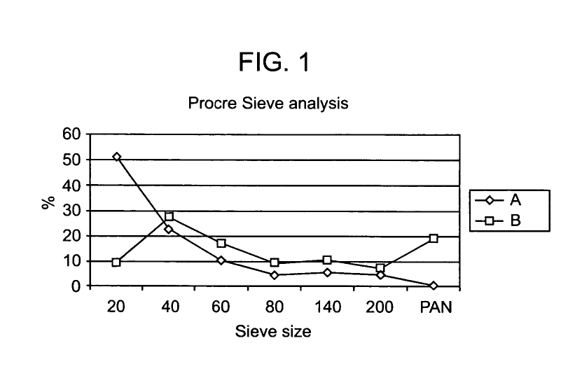

The formulations “A” and “B” were subjected to sieve analysis in a procresieve to obtain the results shown in the graph of FIG. 1.

It was found that by creating a formulation which had a high percentage of particles with a sieve size 20 and the remainder of the particles being in a range of 40 to 60 two desirable characteristics were obtained. Specifically, the granulated particles were better able to be poured or have a “flow” characteristic similar to that of sand flowing through an hourglass. However, when the particles did not have the desired particle size range they did not have the desired “flow” characteristic. Further, when the formulation was created to have the desired particle size range the formulation could be more readily formed into tablets. It is undesirable to have particles which are either too small or too large in that such could interfere with both the “flow” characteristics and the ability to create tablets with the particles. In accordance with the invention 40 to 60% and more preferably about 50%±5% of the particles should have a sieve size of about 20. 10 to 30% of the particles or more preferably 20%+5% should have a sieve size of about 40 and 5 to 15% of the particles should have a sieve size of about 60 or more preferably 10%±5% of the particles should have a sieve size of 60. The remainder of the particles should have a sieve size of either less than 20 or greater than 60 and these small or larger particles should constitute 10% or less of the formulation.

### Therapeutic Blood Levels

One aspect of the invention is that a range of highly desirable therapeutic effects are obtained even when the creatine blood serum levels are maintained in a range well below those previous used. The present invention could obtain desired therapeutics effects with higher levels of creatine in blood serum. However, at least minimum levels would need to be constantly maintained over a long period of time (4 hours or more per day) for a plurality of days to obtain the desired results. When the oral dosage form is designed to obtain the lowest possible therapeutic level over the longest possible time period the results obtained are maximized and the amount of drug needed is minimized.

The creatine blood plasma level obtained via the present invention is insufficient to obtain a desired therapeutic effect if that level is maintained for only a short period of time, e.g., 4 hours or less. However, by using the controlled release formulation of the invention these lower creatine blood plasma levels can be maintained over 8 hours or more, preferably over 12 hours or more and more preferably over 16 hours or more per day. Further, those creatine blood plasma levels over these periods of time are repeatedly obtained over a period of days, preferably weeks or months and more preferably continuously over any period during which the patient would benefit from, for example, the substance's ability to enhance muscle performance—which may be the remainder of the patient's life.

To obtain the desired results, a formulation of the invention includes a sufficient amount of creatine such that it is capable of releasing enough creatine per unit of time to obtain the desired creatine serum levels while compensating for creatine which is metabolized. To obtain the desired results the formulation may immediately and quickly provide an initial release of creatine and thereafter provide a gradual release which slows over the useful life of the formulation. However, the release may be gradual from the beginning. In either case, there is a gradual slowing of the rate of release which is compensated for in that some of the previously released creatine remains in the blood serum unmetabolized.

Creatine is biologically active up until it becomes creatinine as shown below:

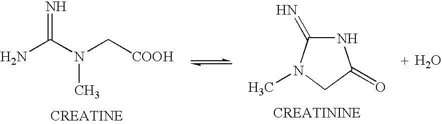

The half-life of creatine in blood plasma is short (1-1.5 hours). This makes it necessary to reach high blood plasma levels rapidly. In view of the bioavailability of creatine, such blood plasma levels can be obtained only by the administration of high doses of creatine, e.g. 5-10 g for mean body weights of about 70 kg. Such high amounts are well tolerated because the toxicity of creatine is quite low.

A creatine ester will maintain its structure in the stomach and intestines. The creatine ester becomes creatine in the blood maintaining its biological activity as follows: 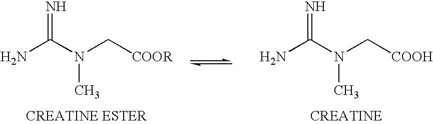
wherein R is an alkyl e.g. ethyl.

As indicated above the active creatine will eventually become the inactive creatinine.

A preferred oral formulation is a tablet which is designed to dissolve gradually over a period of about 8 hours. As the tablet dissolves, its reduced size will release smaller and smaller amounts of creatine per unit of time. However, because the individuals system already contains a therapeutic level of creatine, the slower release rate is sufficient to match the rate of creatine being metabolized and such will result in maintaining a relatively constant therapeutic level. At the end of the time when release of creatine is no longer taking place (e.g., about 4 to 8 hours), another tablet is administered and the process is repeated. To obtain the benefits of the invention, the process is continually repeated over a plurality of days, weeks, months or years. By maintaining a minimal creatine blood serum level over time, a patient's muscle performance is enhanced.

### Excipient Material

Examples provided here show that formulations of the invention may comprise different amounts and ratios of active ingredient and excipient material. Further, different excipients can be used. Particularly preferred excipients and amounts used are recited in the Examples. However, upon reading the disclosure those skilled in the art will come to understand the general concepts of the invention and will recognize that other excipients, amounts, ratios and combinations might be used to obtain the results first shown here.

Some of the preferred excipient materials for use in formulations of the invention are dicalcium phosphate which may be present in an amount in the range of from about 15 to 5%; polyvinyl pyrolidine which may be present in a range of from about 1% to 5%; starch which may be present in an amount in a range of about 1% to 5% and magnesium stearate which may be present in an amount of 0.5% to 2% with all percent amounts being percent by weight based on the total weight of the composition. The remainder of the composition would be the active ingredient which is a creatine derivative and preferably a creatine ester and most preferably creatine ethyl ester.

After the excipients are added to the active ingredient the formulation is ground to obtain the desired particle size as described above. The particles are then preferably coated using a shellac which is used to mask the bitter flavor of the creatine ester material. A salt in base solution of up to 10% by weight may be used to coat the particles. The shellac may be maintained on the particles in an amount in a range of about 1% to 7.5% or 1/5% to 5%; or 2.5 weight percent of the total particle weight±10%. The shellac coating aids in not only masking the flavor but in preventing water absorption by the particles which can reduce the shelf life and inactivate the creatine ester.

The type and amount of excipient material is added to obtain a formulation having certain characteristics. First, the resulting formulation protects the active ingredient from chemical degradation in the patient's gastrointestinal tract. A formulation of pure, unprotected creatine or creatine ester is not part of the scope of the present invention in that pure creatine or creatine ester is degraded to some degree in the gastrointestinal tract. Although the formulation need not protect 100% of the creatine or creatine ester from degradation to come within the scope of the invention, it should protect at least 90% or more, preferably 95% or more and more preferably 99% or more of the creatine or creatine ester from degradation. Although multiple doses of an oral formulation could be taken it is preferable to design the dosage such that a single dose is taken at each dosing event—preferably three times a day and more preferably twice a day. The better the active ingredient is protected from degradation the less active ingredient is needed in the original dosage thereby reducing manufacturing costs and increasing profits. The formulation must protect at least as much of the dose as is needed to obtain a pharmacological effect and preferably obtain the desired treatment results, e.g., maintaining a desired creatine serum level needed to obtain desired results.

Another characteristic of the formulation is that it does not release all of the active ingredient at one time but rather releases the active ingredient gradually over time at a controlled rate of release which rate is preferably constant over 4 hours or more. This is particularly important because a desired level of creatine in blood serum should be maintained over a long period to obtain the desired effect. If all of the creatine is released at once, it will all enter the circulatory system at once and be metabolized to creatinine causing the creatine serum level to drop below the desired level. When this occurs, any effect on enhancing muscle performance would be suboptimal.

### Methods of Treatment

Formulations of the invention may be administered to patients having myoclonus (i.e., a neuromuscular disorder characterized by the occurrence of irregular, asynergic, and jactitious contractions of muscles producing non repetitive, brief, involuntary movements in various body areas) as a symptom of epilepsy, neurodegenerative disease such as Parkinson's disease, multiple sclerosis or amyotrophic lateral sclerosis (ALS) and Tourette's syndrome.

There are several metabolic diseases of human and animal metabolism, e.g., obesity and severe weight loss that relate to energy imbalance—where caloric intake versus energy expenditure—is imbalanced. Obesity, which can be defined as a body weight more than 20% in excess of the ideal body weight, is a major health problem in Western affluent societies. It is associated with an increased risk for cardiovascular disease, hypertension, diabetes, hyperlipidaemia and an increased mortality rate. Obesity is the result of a positive energy balance, as a consequence of an increased ratio of caloric intake to energy expenditure.

The creatine kinase/creatine phosphate system is an energy generating system operative predominantly in the brain, muscle, heart, retina, adipose tissue and the kidney (Walliman et. al., Biochem. J. 281: 21-40 (1992)). The components of the system include the enzyme creatine kinase (CK), the substrates creatine (Cr), creatine phosphate (CrP), ATP, ADP, and the creatine transporter. The enzyme catalyzes reversibly the transfer of a phosphoryl group from CrP to ADP to generate ATP which is the main source of energy in the cell. This system represents the most efficient way to generate energy upon rapid demand. The creatine kinase isoenzymes are found to be localized at sites where rapid rate of ATP replenishment is needed such as around ion channels and ATPase pumps. Some of the functions associated with this system include efficient regeneration of energy in the form of ATP in cells with fluctuating and high energy demand, energy transport to different parts of the cell, phosphoryl transfer activity, ion transport regulation, and involvement in signal transduction pathways.

The substrate Cr is a compound which is naturally occurring and is found in mammalian brain, skeletal muscle, retina, adipose tissue and the heart. The phosphorylated form of Cr, CrP, is also found in the same organs and is the product of the CK reaction. Both compounds can be easily synthesized and are believed to be non toxic to man. A series of creatine analogues have also been synthesized and used as probes to study the active site of the enzyme. Kaddurah-Daouk et al. (WO 92/08456 published May 29, 1992 and WO 90/09192, published Aug. 23, 1990; U.S. Pat. No. 5,321,030; and U.S. Pat. No. 5,324,731) described methods for inhibiting growth, transformation, or metastasis of mammalian cells using related compounds. Examples of such compounds include cyclocreatine, homocyclocreatine and beta guanidino propionic acid.

It is an object of the present invention to provide methods for treatment of metabolic diseases that relate to deregulated body weight by administering to an afflicted individual a creatine derivative formulation which modulates one or more of the structural or functional components of the creatine kinase/creatine phosphate system sufficient to prevent, reduce or ameliorate the symptoms of the disease.

Formulations of creatine derivatives of the invention can be used in methods of treating muscle degeneration and weakness. More particularly, the present invention relates to oral administration of a formulation of an ethyl ester of creatine for the treatment of muscle degeneration and weakness.

Progressive degeneration and weakness of skeletal muscles are hallmarks of the forty human neuromuscular diseases affecting motoneurones, peripheral nerves and/or muscles. Most of these diseases are fatal, and all are crippling. There is no known cure or effective treatment. These diseases include motoneurone disorders, such as Amyotrophic Lateral Sclerosis (ALS) and neuromuscular junction disorders, such as Myasthenia Gravis and Eaton-Lambert Syndrome. Also included are the twelve hereditary muscular dystrophies, predominantly muscle diseases, affecting over 200,000 Americans. In the muscular dystrophies, dystrophic cells degenerate because of the lack of normal genome.

Muscular dystrophy in the mouse is characterized by progressive degeneration of skeletal muscles in the hindlimbs and in the chest wall. Dystrophic symptoms first appear at 20 to 30 days after birth and consist of sporadic flexion and flaccid extension of the hindlimbs. Occasionally, the dystrophic mouse walks with duck feet (See for example, Michelson et al., Proc. Nat. Acad. Sci., 41: 10798, (1955) and Meier et al., Life Sci., 9: 137, (1970)). A number of approaches have been employed by researchers in the field to study and develop methods to treat the muscular dystrophies and other neuromuscular disorders.

In the case of the hereditary neuromuscular disorders, one approach to correct the genetic disease is to correct the abnormal gene itself. However, before gene therapy can be used to treat hereditary myopathies, the defective genes and their expression have to be determined. Although identification of the dystrophic genes and their primary protein abnormalities has been attempted by some workers, thus far, attempts at identification have not been completely successful. (See e.g., Monaco et al., Nature 323: 646-650, 1986; Brown et al., Hum. Genet. 71: 62-74, 1985). Furthermore, before gene therapy can be used to treat hereditary myopathies, the problems of nonspecific gene integration, replacement, targeting, regulation and expression also have to be overcome. The high spontaneous mutation rate also complicates the process of identification and prevention. (See e.g., Epstein et al., Am Sci 65: 703-711, 1977.) When normal and dystrophic tissues are compared, the dystrophy-specific protein difference is often masked by the concomitant presence of individual-specific protein differences (see, e.g., Komi et al., Acta. Physiol. Scand. 100:385-392, 1977) and secondary degenerative changes (See, e.g., Dolan et al., Exp. Neurol. 47:105-117, 1975).

In Duchenne muscular dystrophy, carrier detection and prenatal diagnosis seek prevention rather than cure. See, e.g., Bechmann, Isr. J. Med Sci 13:102-106, 1977. These are inadequate measures, because not all sex-linked carriers—inasmuch as they are phenotypically normal—are exposed to the diagnostic tests.

Various studies have been carried out in attempts to develop methods to treat neuromuscular disease.

In one reported approach, mouse muscle mince transplants studies were conducted on normal and dystrophic littermates (Law, Exp. Neurol., 60:231, 1978). In another study, it is reported that near-normal contractile properties were produced in adult dystrophic mouse muscle by grafting a muscle of a newborn normal mouse into a recipient muscle of a dystrophic mouse (Law et al., Muscle & Nerve, 2:356, 1979). It is also been reported that mesenchyme transplantation can improve the structure and function of dystrophic mouse muscle as demonstrated by histological, electrophysiological and mechanophysiological studies (Law, Muscle & Nerve, 5:619, 1982).

Various attempts have been made to provide treatments for neuromuscular disorders. However, none have achieved recovery of muscle function, locomotive pattern and respiratory function in a host affected with muscle degeneraion and weakness. The compositions and methods of treating such disorders with formulations of the invention are provided.

### Examples of Formulations

A typical formulation of the invention will contain about 70% to about 90% by weight of creatine ester (or some other derivative of creatine) and a particularly preferred formulation will comprise 80%±5% by weight of creatine ester. Assuming a formulation with about 80% by weight of creatine ester with the remaining being excipient material, there are a number of possible components which could be used to make up the remainder of the formulation A generalized and specific description of such is provided below:

| | | |
|---|---|---|
| (1) | Creatine ester | 80% |
| | biodegradable polymer | 20% |
| | TOTAL | 100% |
| (2) | Creatine ester | 80% |
| | biodegradable polymer | 14.5% |
| | Inorganics | 5.5% |
| | TOTAL | 100% |
| (3) | creatine ester | 80% |
| | organic polymer | 10%-20% |
| | Inorganics | 10% or less |
| | TOTAL | 100% |
| (4) | creatine ester | 80% |
| | microcrystalline cellulose | 4% |
| | Cellulose acetate phthalate aqueous | 5% |
| | dispersion | |
| | Polyvinylpyrolidone | 3% |
| | ethyl acetate | 2.5% |
| | hydrous magnesium silicate (talc) | 1% |
| | carboxy methyl ether | 4% |
| | magnesium stearate | 0.5% |
| | TOTAL | 100% |
| (5) | creatine ester | 80% |
| | microcrystalline cellulose | 5-20% |
| | Cellulose acetate phthalate aqueous | 5-15% |
| | dispersion | |
| | polyvinylpyrolidone | 1-5% |
| | ethyl acetate | 1-5% |
| | hydrous magnesium silicate (talc) | 0.5-3% |
| | carboxy methyl ether | 1-5% |
| | magnesium stearate | 0.5-1.5% |
| | TOTAL | 100% |
| (6) | creatine ester | 70% |
| | microcrystalline cellulose, NF (Avicel PH | 14% |
| | 101) | |
| | Aquacoat CPD-30 (30% solids w/w) | 5% |
| | Plasdone K29/32, USP | 3% |
| | Carbopol 974P, NF | 2.5% |
| | Talc, USP | 1.0% |
| | croscarmellose sodium, NF (Ac, di-Sol) | 4.0% |
| | Magnesium Stearate, NF | 0.5% |
| | TOTAL | 100% |
| (7) | creatine ethyl ester | 75%-85% |
| | Diacalcium phosphate | 5-15% |
| | polyvinyl pyrrolidone | 2-4% |
| | Starch | 2-4% |
| | Magnesium Stearate, NF | 0.5-1.5% |
| | TOTAL | 100% |
| (8) | creatine ethyl ester | 83% |
| | Di-Calcium Phosphate | 10% |
| | polyvinyl pyrrolidone (Kollidon 90) | 3% |
| | Starch | 3% |
| | Magnesium stearate | 1% |
| | TOTAL | 100% |
| (9) | creatine ethyl ester | 80% |
| | Poly-DL-lactide-co-glycolide (PLG) | 20% |
| | TOTAL | 100% |
| (10) | creatine ethyl ester | 70% |
| | hydroxypropyl methylcellulose | 20% |
| | Spray-dried lactose | 9.5% |
| | Magnesium stearate | 0.5% |
| | TOTAL | 100% |
| (11) | creatine ethyl ester | 70-75% |
| | polyvinyl pyrrolidone (Kollidon 90) | 10-20% |
| | Lactose | 5-15% |
| | microcrystalline cellulose | 4-6% |
| | titanium dioxide | 1-5% |
| | TOTAL | 100% |
| (12) | creatine ethyl ester | 80% |
| | polyvinyl pyrrolidone (Kollidon 90) | 20% |
| | TOTAL | 100% |
| (13) | creatine ethyl ester | 80% |
| | polyvinyl pyrrolidone | 5% |
| | D calcium phosphate | 15% |
| | TOTAL | 100% |
| (14) | creatine ethyl ester | 83% |
| | polyvinyl pyrrolidone | 5% |
| | D calcium phosphate | 12% |
| | TOTAL | 100% |
| (15) | creatine ethyl ester | 75% |
| | polyvinyl pyrrolidone | 5% |
| | dibasic calcium phosphate | 15% |
| | Starch | 5% |
| | TOTAL | 100% |
| (16) | creatine ethyl ester | 75-85% |
| | hydroxyalkylcellulose | 10-20% |
| | Lactose | 5-10% |
| | microcrystalline cellulose | 4-6% |
| | titanium dioxide | 1-5% |
| | TOTAL | 100% |
| (17) | creatine ethyl ester | 80% |
| | Alkylcellulose | 10% |
| | spray-dried lactose | 9.5% |
| | magnesium stearate | 0.5% |
| | TOTAL | 100% |
| (18) | creatine ethyl ester | 80% |
| | carboxymethylcellulose (hydrogel matrix) | 10% |
| | polyethylene oxide (hydrogel matrix) | 10% |
| | TOTAL | 100% |
| (19) | creatine ethyl ester | 80% |
| | polyvinylpyrrolidone (hydrogel matrix) | 5% |
| | polyethylene glycol (hydrogel matrix) | 15% |
| | TOTAL | 100% |
| (20) | creatine ethyl ester | 70-80% |
| | hydroxypropyl methylcellulose | 5-10% |
| | Ethylcellulose | 5-10% |
| | Lactose | 5-15% |
| | Sorbitol | 4-6% |
| | silicon dioxide | 1-5% |
| | TOTAL | 100% |
| (21) | creatine ethyl ester | 80% |
| | cellulose acetate butyrate | 10% |
| | Starch | 9.5% |
| | magnesium stearate | 0.5% |
| | TOTAL | 100% |
| (22) | creatine ethyl ester | 70% |
| | cellulose acetate phthalate | 10% |
| | cellulose acetate trimellitate | 10% |
| | Mannitol | 9.5% |
| | calcium stearate | 0.5% |
| | TOTAL | 100% |
| (23) | creatine ethyl ester | 80% |
| | polyvinylacetate phthalate | 5% |
| | hydroxypropylmethylcelluulose phthalate | 5% |
| | Sucrose | 5-9% |
| | stearic acid | 1-5% |
| | TOTAL | 100% |
| (24) | creatine ethyl ester | 80% |
| | Methylcellulose | 10% |
| | hydroxypropylmethylcellulose | 5% |
| | Glucose | 4% |
| | Talc | 0.5% |
| | PEG 6000 | 0.5% |
| | TOTAL | 100% |
| (25) | creatine ethyl ester | 70% |
| | polyethylene glycol | 10% |
| | poly(alkyl methacrylate) | 10% |
| | calcium stearate | 5% |
| | dibasic calcium phosphate | 3% |
| | Poloxamers | 2% |
| | TOTAL | 100% |
| (26) | creatine ethyl ester | 80% |
| | Hydroxypropylmethylcellulose | 14% |
| | Pectin | 12% |
| | magnesium stearate | 4% |
| | TOTAL | 100% |
| (27) | creatine ethyl ester | 76.7% |
| | calcium sulfate | 7.3% |
| | Zein | 1.3% |
| | Alginate | 3.3% |
| | Pectin | 4.0% |
| | Glycerin | 6.7% |
| | magnesium stearate | 0.7% |
| | TOTAL | 100% |

Oral dosage units comprising a creatine derivative are judged by many as having bitter favor. Thus, it is desirable to mask such which can be done by coating the dosage (e.g. tablet) with a dissolvable coating. Such a coating may be a pharmaceutical grade shellac or like material. The coating may add an additional 1% to 4% by weight to the dosage unit.

Those skilled in the art will recognize that there are endless possibilities in terms of formulations and that a margin of error e.g., ±20% or more preferably ±10%, should be accounted for with each component. Even if the formulations are limited to the relatively few compounds shown above, the formulation could be changed in limitless ways by adjusting the ratios of the components to each other. A feature of an embodiment of a formulation of the invention is that the creatine ester be released in a controlled manner which makes it possible to maintain therapeutic levels of creatine over a substantially longer period of time as compared to a quick release formulation or with a creatine formulation. A particularly preferred formulation will quickly obtain a therapeutic level and thereafter decrease the rate of release to closely match the rate at which creatine ester becomes creatine thereby maintaining a therapeutic level in the patient over a maximum period of time based on the amount of creatine ester in the oral dosage formulation. Some general types of controlled release technology which might be used with the present invention are described below followed by specific preferred formulations.

Formulations of the invention as described above are a “quick release” formulations of creatine derivative and such provides a number of advantages as compared to formulations of creatine. The creatine derivatives formulated in accordance with the present invention provide improved bioavailability as compared with creatine formulations. That improved bioavailability provides improved results in a number of areas as described here. However, formulations of the invention can be created so as to provide sustained release or controlled release of the active ingredient. When the active ingredient is maintained at therapeutic levels over longer periods of time results obtained are improved. Accordingly, the following provides information relating to the production of controlled release formulations.

### Controlled Release Technology

Controlled release within the scope of this invention can be taken to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

There are companies with specific expertise in drug delivery technologies including controlled release oral formulations such as Alza Corporation and Elan Pharmaceuticals, Inc. A search of patents, published patent applications and related publications will provide those skilled in the art reading this disclosure with significant possible controlled release oral formulations. Examples include the formulations disclosed in any of the U.S. Pat. No. 5,637,320 issued Jun. 10, 1997; U.S. Pat. No. 5,505,962 issued Apr. 9, 1996; U.S. Pat. No. 5,641,745 issued Jun. 24, 1997; and U.S. Pat. No. 5,641,515 issued Jun. 24, 1997. Although specific formulations are disclosed here and in these patents, the invention is more general than any specific formulation. This includes the discovery that by placing creatine esters in a controlled release formulation which maintains therapeutic levels over substantially longer periods of time, as compared to quick release formulations, improved unexpected results are obtained.

The various controlled release technologies cover a very broad spectrum of drug dosage forms. Controlled release technologies include, but are not limited to physical systems and chemical systems.

Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, Controlled Release Technologies: Methods, Theory and Applications, 1980 (CRC Press, Inc.).

Controlled release drug delivery systems may also be categorized under their basic technology areas, including, but not limited to, rate-preprogrammed drug delivery systems, activation-modulated drug delivery systems, feedback-regulated drug delivery systems, and site-targeting drug delivery systems.

In rate-preprogrammed drug delivery systems, release of drug molecules from the delivery systems “preprogrammed” at specific rate profiles. This may be accomplished by system design, which controls the molecular diffusion of drug molecules in and/or across the barrier medium within or surrounding the delivery system. Fick's laws of diffusion are often followed.

In activation-modulated drug delivery systems, release of drug molecules from the delivery systems is activated by some physical, chemical or biochemical processes and/or facilitated by the energy supplied externally. The rate of drug release is then controlled by regulating the process applied, or energy input.

In feedback-regulated drug delivery systems, release of drug molecules from the delivery systems may be activated by a triggering event, such as a biochemical substance, in the body. The rate of drug release is then controlled by the concentration of a triggering agent detected by a sensor in the feedback regulated mechanism.

In a site-targeting controlled-release drug delivery system, the drug delivery system targets the active molecule to a specific site or target tissue or cell. This may be accomplished, for example, by a conjugate including a site specific targeting moiety that leads the drug delivery system to the vicinity of a target tissue (or cell), a solubilizer that enables the drug delivery system to be transported to and preferentially taken up by a target tissue, and a drug moiety that is covalently bonded to the polymer backbone through a spacer and contains a cleavable group that can be cleaved only by a specific enzyme at the target tissue.

While a preferable mode of controlled release drug delivery will be oral, other modes of delivery of controlled release compositions according to this invention may be used. These include mucosal delivery, nasal delivery, ocular delivery, transdermal delivery, parenteral controlled release delivery, vaginal delivery, and intrauterine delivery.

There are a number of controlled release drug formulations that are developed preferably for oral administration. These include, but are not limited to, osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-release gastrointestinal delivery devices; gel diffusion-controlled gastrointestinal delivery systems; and ion-exchange-controlled gastrointestinal delivery systems, which include cationic and anionic drugs. Additional information regarding controlled release drug delivery systems may be found in Yie W. Chien, Novel Drug Delivery Systems, 1992 (Marcel Dekker, Inc.). Some of these formulations will now be discussed in more detail.

Enteric coatings are applied to tablets to prevent the release of drugs in the stomach either to reduce the risk of unpleasant side effects or to maintain the stability of the drug which might otherwise be subject to degradation due to exposure to the gastric environment. Most polymers that are used for this purpose are polyacids that function by virtue or the fact that their solubility in aqueous medium is pH-dependent, and they require conditions with a pH higher then that which is normally encountered in the stomach.

One preferable type of oral controlled release structure is enteric coating of a solid or liquid dosage form. Enteric coatings promote the drug or other compound remaining physically incorporated in the dosage form for a specified period when exposed to gastric juice. Yet the enteric coatings are designed to disintegrate in intestinal fluid for ready absorption. Delay of the drug or other compound's absorption is dependent on the rate of transfer through the gastrointestinal tract, and so the rate of gastric emptying is an important factor. Some investigators have reported that a multiple-unit type dosage form, such as granules, may be superior to a single-unit type. Therefore, in a preferable embodiment, the creatine esters may be contained in an enterically coated multiple-unit dosage form. In a more preferable embodiment, the creatine esters dosage form is prepared by spray-coating granules of a creatine esters-enteric coating agent solid dispersion on an inert core material. These granules can result in prolonged absorption of the drug with good bioavailability.

Typical enteric coating agents include, but are not limited to, hydroxypropylmethylcellulose phthalate, methacryclic acid-methacrylic acid ester copolymer, polyvinyl acetate-phthalate and cellulose acetate phthalate. Akihiko Hasegawa, Application of solid dispersions of Nifedipine with enteric coating agent to prepare a sustained-release dosage form, Chem. Pharm. Bull. 33: 1615-1619 (1985). Various enteric coating materials may be selected on the basis of testing to achieve an enteric coated dosage form designed ab initio to have a preferable combination of dissolution time, coating thicknesses and diametral crushing strength. S. C. Porter et al., The Properties of Enteric Tablet Coatings Made From Polyvinyl Acetate-phthalate and Cellulose acetate Phthalate, J. Pharm. Pharmacol. 22:42p (1970).

On occasion, the performance of an enteric coating may hinge on its permeability. S. C. Porter et al., The Permeability of Enteric Coatings and the Dissolution Rates of Coated Tablets, J. Pharm. Pharmacol. 34: 5-8 (1981). With such oral drug delivery systems, the drug release process may be initiated by diffusion of aqueous fluids across the enteric coating. Investigations have suggested osmotic driven/rupturing affects as important release mechanisms from enteric coated dosage forms. Roland Bodmeier et al., Mechanical Properties of Dry and Wet Cellulosic and Acrylic Films Prepared from Aqueous Colloidal Polymer Dispersions used in the Coating of Solid Dosage Forms, Pharmaceutical Research, 11: 882-888 (1994).

Another type of useful oral controlled release structure is a solid dispersion. A solid dispersion may be defined as a dispersion of one or more active ingredients in an inert carrier or matrix in the solid state prepared by the melting (fusion), solvent, or melting-solvent method. Akihiko Hasegawa, Super Saturation Mechanism of Drugs from Solid Dispersions with Enteric Coating Agents, Chem. Pharm. Bull. 36: 4941-4950 (1998). The solid dispersions are also referred to as solid-state dispersions. The term “coprecipitates” may also be used to refer to those preparations obtained by the solvent methods.

Solid dispersions may be used to improve the solubilities and/or dissolution rates of poorly water-soluble lipoates. Hiroshi Yuasa, et al., Application of the Solid Dispersion Method to the Controlled Release Medicine. III. Control of the Release Rate of Slightly Water-Soluble Medicine From Solid Dispersion Granules, Chem. Pharm. Bull. 41:397-399 (1993). The solid dispersion method was originally used to enhance the dissolution rate of slightly water-soluble medicines by dispersing the medicines into water-soluble carriers such as polyethylene glycol or polyvinylpyrolidone, Hiroshi Yuasa, et al., Application of the Solid Dispersion Method to the Controlled Release of Medicine. IV. Precise Control of the Release Rate of a Water-Soluble Medicine by Using the Solid Dispersion Method Applying the Difference in the Molecular Weight of a Polymer, Chem. Pharm. Bull. 41:933-936 (1993).

The selection of the carrier may have an influence on the dissolution characteristics of the dispersed drug because the dissolution rate of a component from a surface may be affected by other components in a multiple component mixture. For example, a water-soluble carrier may result in a fast release of the drug from the matrix, or a poorly soluble or insoluble carrier may lead to a slower release of the drug from the matrix. The solubility of the creatine esters may also be increased owing to some interaction with the carriers.

Examples of carriers useful in solid dispersions according to the invention include, but are not limited to, water-soluble polymers such as polyethylene glycol, polyvinylpyrolidone, or hydroxypropylmethyl-cellulose. Akihiko Hasegawa, Application of Solid Dispersions of Nifedipine with Enteric Coating Agent to Prepare a Sustained-release Dosage Form, Chem. Pharm. Bull. 33: 1615-1619 (1985).

Alternate carriers include phosphatidylcholine. Makiko Fujii, et al., The Properties of Solid Dispersions of Indomethacin, Ketoprofen and Flurbiprofen in Phosphatidylcholine, Chem. Pharm. Bull. 36:2186-2192 (1988). Phosphatidylcholine is an amphoteric but water-insoluble lipid, which may improve the solubility of otherwise insoluble creatine esters in an amorphous state in phosphatidylcholine solid dispersions. See Makiko Fujii, et al., Dissolution of Bioavailibility of Phenyloin in Solid Dispersion with Phosphatidylcholine, Chem. Pharm. Bull 36:49084913 (1988).

Other carriers include polyoxyethylene hydrogenated castor oil. Katsuhiko Yano, et al., In-Vitro Stability and In-Vivo Absorption Studies of Colloidal Particles Formed From a Solid Dispersion System, Chem. Pharm. Bull 44:2309-2313 (1996). Poorly water-soluble creatine esters may be included in a solid dispersion system with an enteric polymer such as hydroxypropylmethylcellulose phthalate and carboxymethylethylcellulose, and a non-enteric polymer, hydroxypropylmethylcellulose. See Toshiya Kai, et al., Oral Absorption Improvement of Poorly Soluble Drug Using Soluble Dispersion Technique, Chem. Pharm. Bull. 44:568-571 (1996). Another solid dispersion dosage form includes incorporation of the drug of interest with ethyl cellulose and stearic acid in different ratios. Kousuke Nakano, et al., Oral Sustained-Release Cisplatin Preparations for Rats and Mice, J. Pharm. Pharmacol. 49:485-490 (1997).

There are various methods commonly known for preparing solid dispersions. These include, but are not limited to the melting method, the solvent method and the melting-solvent method.

In the melting method, the physical mixture of a drug in a water-soluble carrier is heated directly until it melts. The melted mixture is then cooled and solidified rapidly while rigorously stirred. The final solid mass is crushed, pulverized and sieved. Using this method a super saturation of a solute or drug in a system can often be obtained by quenching the melt rapidly from a high temperature. Under such conditions, the solute molecule may be arrested in solvent matrix by the instantaneous solidification process. A disadvantage is that many substances, either drugs or carriers, may decompose or evaporate during the fusion process at high temperatures. However, this evaporation problem may be avoided if the physical mixture is heated in a sealed container. Melting under a vacuum or blanket of an inert gas such as nitrogen may be employed to prevent oxidation of the drug or carrier.

The solvent method has been used in the preparation of solid solutions or mixed crystals of organic or inorganic compounds. Solvent method dispersions may prepared by dissolving a physical mixture of two solid components in a common solvent, followed by evaporation of the solvent. The main advantage of the solvent method is that thermal decomposition of drugs or carriers may be prevented because of the low temperature required for the evaporation of organic solvents. However, some disadvantages associated with this method are the higher cost of preparation, the difficulty in completely removing liquid solvent, the possible adverse effect of its supposedly negligible amount of the solvent on the chemical stability of the drug.

Another method of producing solid dispersions is the melting-solvent method. It is possible to prepare solid dispersions by first dissolving a drug in a suitable liquid solvent and then incorporating the solution directly into a melt of polyethylene glycol, obtainable below 70 degrees, without removing the liquid solvent. The selected solvent or dissolved drug may be selected such that the solution is not miscible with the melt of polyethylene glycol. The polymorphic form of the drug may then be precipitated in the melt. Such a unique method possesses the advantages of both the melting and solvent methods. Win Loung Chiou, et al., Pharmaceutical Applications of Solid Dispersion Systems, J. Pharm. Sci. 60:1281-1301 (1971).

Another controlled release dosage form is a complex between an ion exchange resin and the drug. Ion exchange resin-drug complexes have been used to formulate sustained-release products of acidic and basic drugs. In one preferable embodiment, a polymeric film coating is provided to the ion exchange resin-drug complex particles, making drug release from these particles diffusion controlled. See Y. Raghunathan et al., Sustained-released drug delivery system I: Coded ion-exchange resin systems for phenylpropanolamine and other drugs, J. Pharm. Sciences 70: 379-384 (1981).

Injectable micro spheres are another controlled release dosage form. Injectable micro spheres may be prepared by non-aqueous phase separation techniques, and spray-drying techniques. Micro spheres may be prepared using polylactic acid or copoly(lactic/glycolic acid). Shigeyuki Takada, Utilization of an Amorphous Form of a Water-Soluble GPIlb/IIIa Antagonist for Controlled Release From Biodegradable Micro spheres, Pharm. Res. 14:1146-1150 (1997), and ethyl cellulose, Yoshiyuki Koida, Studies on Dissolution Mechanism of Drugs from Ethyl Cellulose Microcapsules, Chem. Pharm. Bull. 35:1538-1545 (1987).

Other controlled release technologies that may be used in the practice of this invention are quite varied. They include SODAS, INDAS, IPDAS, MODAS, EFVAS, DUREDAS. SODAS are multi particulate dosage forms utilizing controlled release beads. INDAS are a family of drug delivery technologies designed to increase the solubility of poorly soluble drugs. IPDAS are multi particulate tablet formation utilizing a combination of high density controlled release beads and an immediate release granulate. MODAS are controlled release single unit dosage forms. Each tablet consists of an inner core surrounded by a semipermeable multiparous membrane that controls the rate of drug release. EFVAS is an effervescent drug absorption system. PRODAS is a family of multi particulate formulations utilizing combinations of immediate release and controlled release mini-tablets. DUREDAS is a bilayer tablet formulation providing dual release rates within the one dosage form. Although these dosage forms are known to one of skill, certain of these dosage forms will now be discussed in more detail.

INDAS was developed specifically to improve the solubility and absorption characteristics of poorly water soluble drugs. Solubility and, in particular, dissolution within the fluids of the gastrointestinal tract is a key factor in determining the overall oral bioavailability of poorly water soluble drug. By enhancing solubility, one can increase the overall bioavailability of a drug with resulting reductions in dosage. INDAS takes the form of a high energy matrix tablet, production of which is comprised of two distinct steps: the adensosine analog in question is converted to an amorphous form through a combination of energy, excipients, and unique processing procedures.

Once converted to the desirable physical form, the resultant high energy complex may be stabilized by an absorption process that utilizes a novel polymer cross-linked technology to prevent recrystallization. The combination of the change in the physical state of the drug coupled with the solubilizing characteristics of the excipients employed enhances the solubility of the drug. The resulting absorbed amorphous drug complex granulate may be formulated with a gel-forming erodible tablet system to promote substantially smooth and continuous absorption.

IPDAS is a multi-particulate tablet technology that may enhance the gastrointestinal tolerability of potential irritant and ulcerogenic drugs. Intestinal protection is facilitated by the multi-particulate nature of the IPDAS formulation which promotes dispersion of an irritant drug throughout the gastrointestinal tract. Controlled release characteristics of the individual beads may avoid high concentration of drug being both released locally and absorbed systemically. The combination of both approaches serves to minimize the potential harm of the drug with resultant benefits to patients.

IPDAS is composed of numerous high density controlled release beads. Each bead may be manufactured by a two step process that involves the initial production of a micromatrix with embedded drug and the subsequent coating of this micromatrix with polymer solutions that form a rate limiting semipermeable membrane in vivo. Once an IPDAS tablet is ingested, it may disintegrate and liberate the beads in the stomach. These beads may subsequently pass into the duodenum and along the gastrointestinal tract, preferably in a controlled and gradual manner, independent of the feeding state. Drug release occurs by diffusion process through the micromatrix and subsequently through the pores in the rate controlling semipermeable membrane. The release rate from the IPDAS tablet may be customized to deliver a drug-specific absorption profile associated with optimized clinical benefit. Should a fast onset of activity be necessary, immediate release granulate may be included in the tablet. The tablet may be broken prior to administration, without substantially compromising drug release, if a reduced dose is required for individual titration.

DUREDAS is a bilayer tableting technology that may be used in the practice of the invention. DUREDAS was developed to provide for two different release rates, or dual release of a drug from one dosage form. The term bilayer refers to two separate direct compression events that take place during the tableting process. In a preferable embodiment, an immediate release granulate is first compressed, being followed by the addition of a controlled release element which is then compressed onto this initial tablet. This may give rise to the characteristic bilayer seen in the final dosage form.

The controlled release properties may be provided by a combination of hydrophilic polymers. In certain cases, a rapid release of the drug may be desirable in order to facilitate a fast onset of therapeutic affect. Hence one layer of the tablet may be formulated as an immediate release granulate. By contrast, the second layer of the tablet may release the drug in a controlled manner, preferably through the use of hydrophilic polymers. This controlled release may result from a combination of diffusion and erosion through the hydrophilic polymer matrix.

A further extension of DUREDAS technology is the production of controlled release combination dosage forms. In this instance, two different creatine derivative compounds may be incorporated into the bilayer tablet and the release of drug from each layer controlled to maximize therapeutic affect of the combination.

The creatine esters of the invention can be incorporated into any one of the aforementioned controlled released dosage forms, or other conventional dosage forms. The amount of creatine esters contained in each dose can be adjusted to meet the needs of the individual patient and the indication. One of skill in the art reading this disclosure will readily recognize how to adjust the level of creatine esters and the release rates in a controlled release formulation, in order to optimize delivery of creatine esters and its bioavailability.

### Therapeutic Indications

The controlled release creatine ester formulations of the present invention can be used to obtain a wide range of desirable effects. Formulations of the invention may be administered to patients having myoclonus (i.e., a neuromuscular disorder characterized by the occurrence of irregular, asynergic, and jactitious contractions of muscles producing non repetitive, brief, involuntary movements in various body areas) as a symptom of epilepsy, neurodegenerative disease such as Parkinson's disease, multiple sclerosis or amyotrophic lateral sclerosis (ALS) and Tourette's syndrome. Further, the invention can be used to enhance muscle performance.

Because of the very minimal toxicity of creatine ester, it can be given to a wide range of patients which have different conditions from mild to serious without fear of adverse effects. Further, the controlled release formulations taught here are even safer than quick release formulations in that serum levels obtained are low compared to quick release formulations.

The data provided here do not show specific treatments of many of the diseases or symptoms mentioned above. However, the invention is believed to be responsible for obtaining a wide range of beneficial effects particularly when the controlled release formulation is administered to patients over long periods of time, i.e., weeks, months and years. By maintaining substantially constant therapeutic levels of creatine in the blood over very long periods of time a range of desirable physiological results are obtained. Stated differently, by continually maintaining the constant therapeutic serum levels of creatine muscle performance is enhanced.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. An oral formulation, comprising:
a creatine derivative present in a therapeutically effective amount;
a phosphate; and
a biodegradable polymer.

2. The oral formulation of claim 1, further comprising:
a starch.

3. The oral formulation of claim 1, further comprising:
a metal salt of a stearate.

4. The oral formulation of claim 1, wherein the creatine derivative is an ester.

5. The oral formulation of claim 4, wherein the ester group is —COOR where R is a lower alkyl.

6. The oral formulation of claim 5, wherein R is methyl, ethyl, butyl, isobutyl, or tertiary butyl.

7. A controlled release oral dosage formulation, comprising:
a therapeutically effective amount of a creatine derivative; and
an excipient material; wherein the formulation is characterized by releasing the creatine derivative in a manner so as to increase a period of time over which a therapeutic level of creatine derivative is maintained as compared to a quick release formulation.

8. The formulation of claim 7, wherein the releasing is in a manner which maintains the therapeutic level of creatine in blood for a period of time which is 10% or more longer as compared to a quick release formulation.

9. The formulation of claim 7, wherein the releasing is in a manner which maintains the therapeutic level of creatine in blood for a period of time which is 50% or more longer as compared to a quick release formulation.

10. The formulation of claim 7, wherein the releasing is in a manner which maintains the therapeutic level of creatine in blood for a period of time which is 100% or more longer as compared to a quick release formulation.

11. The formulation of claim 7, wherein the releasing is in a manner which maintains the therapeutic level of creatine in blood for a period of time which is 200% or more longer as compared to a quick release formulation.

12. The formulation of claim 7, wherein the releasing is sufficiently slow that a maximum level of creatine in blood obtained is less as compared to a maximum level obtained with a quick release formulation.

13. The formulation of claim 7, wherein the releasing is sufficiently slow that a maximum level of creatine in blood obtained is 50% or more, less as compared to a maximum level obtained with a quick release formulation.

14. The formulation of claim 7, wherein the releasing of creatine derivative is at a rate of about 25% or less per hour after an initial release rate within 30 minutes following administration as compared to a quick release formulation.

15. The formulation of claim 7, wherein the releasing of creatine derivative is at a rate of about 50% or less per hour after an initial release rate within 30 minutes following administration as compared to a quick release formulation.

16. A method of treating a human patient, comprising:
administering to a human patient a controlled release formulation of creatine derivative which formulation is characterized by maintaining a therapeutic level of creatine in the patient's circulatory system over a period of time greater than that obtained with a quick release formulation; and
repeating the administering on three or more consecutive days thereby maintaining a therapeutic level of creatine in the patient's circulatory system over a therapeutically effective period of time on three or more consecutive days.

17. The method of claim 16, wherein the therapeutic level is maintained over a period of time which is 10% or more than that obtained with a quick release formulation and further wherein the repeating is over thirty or more consecutive days.

18. The method of claim 16, wherein the therapeutic level is maintained over a period of time which is 100% or more than that obtained with a quick release formulation and further wherein the repeating is over thirty or more consecutive days.

19. The method of claim 18, wherein the therapeutic level is a level sufficient to obtain measurable increase in muscle endurance in a human patient.

20. The method of claim 18, wherein the therapeutic level is a level sufficient to enhance muscle performance.

21. An oral formulation, comprising:
a creatine ethyl ester;
a phosphate a biodegradable polymer;
a starch; and
a metal salt of a stearate.

22. The formulation of claim 21, wherein the phosphate is dicalcium phosphate.

23. The formulation of claim 21, wherein the biodegradable polymer is polyvinyl pyrrolidine.

24. The formulation of claim 21, wherein the stearate is magnesium stearates.

25. The formulation of claim 21, wherein the creatine ethyl ester is present in the formulation in an amount in a range of about 83%+10% by weight based of the total weight of the formulation.

26. The formulation of claim 22, wherein the dicalcium phosphate is present in an amount in a range of about 9% to about 11% by weight based on the total weight of the formulation.

27. The formulation of claim 23, wherein the polyvinyl pyrrolidone is present in an amount in a range of about 2% to about 4% by weight based on the total weight of the formulation.

28. The formulation of claim 24, wherein the starch is present in an amount in a range of about 2% to about 4% by weight based on the total weight of the formulation.

29. The formulation of claim 25, wherein the magnesium stearate is present in an amount in a range of about 2% to about 4% by weight based on the total weight of the formulation.

30. The formulation of claim 21, in a form chosen from a tablet, a capsule, and a caplet.

31. The formulation of claim 21, comprised of particles where 60% to 40% by weight of the particles have a sieve size of about 20 and 20% to 40% by weight of the particles have a sieve size of about 40.

32. The formulation of claim 31, wherein the formulation of particles is flowable.

33. The formulation of claim 31, wherein 10% or less of the particles have a sieve size of 80 or more.

34. The formulation of claim 33, wherein 10% or less of the particles have a sieve size of 18 or less.

35. The formulation of claim 21, comprised of particles wherein 50% or the particle±5% have a sieve size of 20 and 20% of the particles±5% have a sieve size of 40 and 10% of the particles±5% have a sieve size of 60.

36. A method of treating muscle tissue of a human patient, comprising:
orally administering to a human patient a controlled release formulation of creatine derivative which formulation is comprised of:
a creatine derivative present in a therapeutically effective amount; and
a biodegradable polymer.
